(19) United States
(12) Patent Application Publication
(10) Pub. No.: US 2022/0015822 A1
(43) Pub. Date: Jan. 20, 2022

(54) SURGICAL HANDHELD DEVICE, INSULATION INSERT FOR A SURGICAL HANDHELD DEVICE, AND METHOD FOR MANIPULATING A SURGICAL HANDHELD DEVICE

(71) Applicant: OLYMPUS WINTER & IBE GMBH, Hamburg (DE)

(72) Inventor: Christian BROCKMANN, Hollenstedt (DE)

(73) Assignee: OLYMPUS WINTER & IBE GMBH, Hamburg (DE)

(21) Appl. No.: 17/379,953

(22) Filed: Jul. 19, 2021

(30) Foreign Application Priority Data
Jul. 17, 2020 (DE) 10 2020 118 965.6

(51) Int. Cl. A61B 18/14

(52) U.S. Cl. CPC A61B 18/149; A61B 2018/00083

(57) ABSTRACT

Surgical handheld devices are used in electrosurgical procedures in urology. For this use, a radiofrequency electric current is applied to an electrode. It is necessary to avoid the electrode coming into electrical contact with the handheld device. Known insulation inserts for electrical insulation can be connected to the handheld device only with difficulty. Moreover, the connection is very unreliable. A surgical handheld device, and an insulation insert which can be connected to a shaft of the handheld device in a safe and easily releasable manner. The insulation insert is of a tubular configuration and is releasably coupled with a proximal end region to a distal end of a tubular shaft of the handheld device. The proximal end region of the insulation insert has at least two resilient fasteners, which each have a latch element pointing into an interior of the insulation insert.

The invention relates to an insulation insert for a surgical handheld device according to the preamble of claim 1. The invention further relates to a surgical handheld device according to claim 10, and to a method according to claim 15 for manipulating a surgical handheld device.

Surgical handheld devices, in particular resectoscopes, of the type in question are used primarily in electrosurgical procedures in urology. These devices are customarily used for resection and vaporization of tissue, e.g. of tissue in the lower urinary tract. To this end, the handheld device, in particular the resectoscope, can have a longitudinally displaceable electrosurgical passage instrument which, after the insertion of the device into the body to be treated, can be advanced with its distal working end out of a distal end of a shaft tube of the handheld device. The electrosurgical passage instrument or the electrode instrument has an electrosurgical electrode at its distal end. This electrode can, for example, comprise the form of a loop or of a button or the like.

For the abovementioned uses, a radiofrequency electric current is applied to the electrode. In doing this, it should be ensured that the electrode does not come into electrical contact with the shaft tube, in particular an outer shaft tube or outer tube, of the handheld device. If such electrical contact were to take place, a short circuit could cause a defect in the device and lead to unforeseeable damage in the body that is to be treated. In order to avoid short circuits of this kind, the handheld devices or resectoscopes usually comprise, at their distal end region, a portion that is made of an electrically insulating material. This portion is conventionally referred to as an insulation tip or also as an insulation insert. The insulation insert can be fastened either to an inner shaft or shaft tube, in which an electrode carrier is guided, or to the outer shaft or outer tube. Since these handheld devices or the resectoscopes are designed for repeated use and therefore have to be regularly sterilized or autoclaved, the insulation insert has to satisfy strict requirements as regards durability and re-usability. The choice of material is therefore generally restricted to relatively expensive high-performance ceramics, for example silicon nitrite. Electrically insulating plastics are used as an alternative to these ceramics. These plastics are in particular thermally stable, so that they do not melt immediately upon contact with the electrode.

The releasable coupling of such insulation inserts to the shaft of the handheld device proves to be problematic. In the case of releasable connections between the insulation insert and the shaft, there is basically the risk of the connection accidentally coming loose during the treatment. This must be avoided in all circumstances, since not only can the device itself sustain damage, the person who is to be treated could also be injured. In known mechanisms for fastening insulation inserts to an inner shaft for example, a compromise always has to be made between a retention force and a release force. On the one hand, the fastening of the insulation insert must be strong enough to avoid accidental release. On the other hand, the person carrying out the treatment must still be able to release the insulation insert from the shaft end by applying a reasonable force. If the fastening mechanism is too strong, then, although the level of safety against accidental release is increased, the operator will have difficulty in releasing the insulation insert for the abovementioned purposes. However, if the insulation insert sits too loosely on the inner shaft, then, as has already been mentioned, there is the danger of accidental release.

The problem addressed by the invention is therefore to make available a surgical handheld device, an insulation insert, and a method for manipulating the surgical handheld device, in which method the insulation insert can be connected to a shaft of the handheld device in a secure and easily releasable manner.

A solution to this problem is described by the features of claim 1. Accordingly, provision is made that an insulation insert for a surgical handheld device, in particular for a resectoscope, is of a tubular configuration and is releasably coupled with a proximal end region to a distal end of a tubular shaft of the handheld device. The proximal end region of the insulation insert has at least two resilient fastening means, which each have a latch element pointing into an interior of the insulation insert. With these at least two fastening means, it is possible to ensure that the insulation insert can be coupled sufficiently firmly to the shaft of the handheld device. Equally, these fastening means can be easily and quickly uncoupled by the operator, if appropriate by using an auxiliary tool. These resilient fastening means permit both fastening of the insulation insert and also release of the insulation insert from the surgical handheld device.

According to the invention, provision is preferably made that two, four, six or more fastening means are arranged lying opposite each other on a circumference of the proximal end region. Alternatively, it is also conceivable that three or more fastening means are arranged at an angle of 120° or at the same angle to each other on a circumference of the proximal end region of the insulation insert. Particularly in the case of a non-concentric or axial form of the insulation insert or of the positioning on the inner shaft, a reliable mechanical connection can be produced by this relative positioning of the fastening means on the circumference of the insulation insert. Even in the case of slight tilting of the insulation insert relative to the shaft, unwanted release can be prevented by the positioning of the fastening means. By this arrangement of the fastening means, it is thus possible to ensure a high degree of safety and, at the same time, straightforward manipulation.

In a further preferred illustrative embodiment of the invention, provision can be made that the fastening means on the circumference of the insulation insert are formed as snap-fit hooks. These snap-fit hooks can have tab-like spring elements, at the free ends of which latch elements are arranged. These tab-like spring elements are formed out from the wall of the proximal end region of the insert. By virtue of their tab-like or elongate design, the spring elements have a free spring excursion in a radial direction with respect to the shaft or the insert. Thus, for connection of the insert to the shaft, the spring elements can first of all be pretensioned outwardly, until the latch elements are locked in complementary openings in the shaft. The tensioning of the spring elements has the effect that, without the effect of an external force, they cannot be moved out of the latched retaining position. To uncouple the insulation insert from the shaft, a force has to be applied that is directed counter to the resilient restoring force of the spring elements. Since this force is relatively low on account of the dimensioning of the material, it can be applied by the operator without any problem.

The spring element preferably has a reduced thickness in relation to the wall of the insulation insert. It is equally conceivable that the wall of the insulation insert, in the proximal end region or in the region of the fastening means or between the fastening means, has a reduced thickness in relation to the wall of the insulation insert, preferably that the wall of the insulation insert in the proximal end region has the same thickness as the fastening means, in particular the spring elements. In this way, a ring-like shoulder is obtained on the inner wall of the insulation insert. This shoulder divides the insulation insert into a region with a reduced wall thickness, in which the fastening means are arranged, and a region of greater wall thickness. It is conceivable that the insulation insert is pushed onto the inner shaft in such a way that this ring-like shoulder comes into contact with the tubular opening of the inner shaft. An optimal relative orientation of the insulation insert and of the shaft is thus ensured. A slipping motion, or a relative tilting or rocking, can thereby be avoided. The thickness of the distal region of the insulation insert is preferably dimensioned such that the transition of the inner wall of the inner shaft to the inner wall of the insulation insert is stepless, i.e. in one plane.

In a further preferred illustrative embodiment of the present invention, provision can be made that the fastening means are arranged in pairs next to each other, and the pairs themselves are arranged lying opposite each other on a circumference of the proximal end region. By the fastening means being arranged in pairs in this way, the safety of the releasable connection can be further improved.

In particular, in a further illustrative embodiment, provision is made that the in particular wedge-shaped latch elements have a height corresponding to the wall thickness of the wall of the insulation insert. In addition, the height of the latch elements is dimensioned such that, in the releasable connection to the inner shaft, they do not protrude into the interior of the inner shaft. The profile or a cross section of the latch elements can present a pointed or flattened or rounded wedge. A rounded or flattened wedge can be advantageous for manually pulling the insulation insert from the inner shaft for cleaning purposes and the like. Equally, a latch element with a triangular cross section or sawtooth cross section provides greater safety against unwanted release. The forces for coupling and retention of the spring elements are also dependent on the shape and dimensioning of the latch elements. By adapting the angles of the latch elements, the forces for coupling and retention of the spring elements can be adjusted independently of each other.

The insulation insert described here is preferably made of a plastic. This plastic is both electrically insulating and thermally stable. It is equally conceivable that the insulation insert is made of a coated metal. The coating of the metallic insulation insert in this case affords sufficient electrical insulation. A metallic embodiment of the insulation insert can be particularly advantageous for a long useful life of the insert.

A surgical handheld device for solving the stated problem has the features of claim 10. The surgical handheld device can in particular be a resectoscope or the like. The claimed surgical handheld device has an inner shaft or shaft tube and an outer shaft or outer tube. The outer tube is arranged around the inner shaft. According to the invention, an insulation insert according to claim 1 is assigned releasably to a distal end of the inner shaft. By this arrangement of the insulation insert at the distal end both of the inner shaft and of the outer shaft, it is possible to prevent a situation where an electrosurgical tool or an electrode, guided through the inner shaft out of the insulation insert, does not come into electrical contact with the metallic shaft tubes. Since the insulation insert is made of an electrically insulating material, contact between the electrosurgical tool and the insulation insert is not critical.

Provision is preferably made that an outer circumference of the proximal end region of the insulation insert is dimensioned in such a way that it can be pushed over a distal end of the inner shaft. Provision can be made here that the wall of the insulation insert has different thicknesses, such that the inner wall of the insert has a ring-like peripheral shoulder which, when pushed onto the inner shaft, comes into contact with the ring-like end of the shaft. In this way, an optimal orientation of the insulation insert on the shaft can be obtained. In addition, the wall of the inner shaft has openings in which the latch elements from the fastening means of the insulation insert engage for a releasable coupling. These openings can be arranged peripherally or opposite each other in the wall of the shaft. These openings can have the shape of circles, rectangles or polyhedrons or ovals. The openings in the wall of the shaft are configured such that they correspond to the shape or to a cross section of the latch elements. The openings in the wall can constitute holes in the wall or simply trough-like recesses in the wall, into which the latch elements can snap.

It is furthermore conceivable that a distance between an inner face of the outer shaft and the outer face of the insulation insert is less than the height of the latch elements of the fastening means of the insulation insert. By virtue of the fact that a ring-like gap at least in the middle between the insulation insert and the outer shaft has a width less than the height of the latch elements, the insulation insert coupled to the inner shaft is secured by the outer shaft. Through this dimensioning of the external diameter of the insulation insert or of the diameter of the outer shaft, accidental release of the fastening means from the openings of the inner shaft is not possible. Even if the fastening means or the latch elements were to be moved by the effect of a force, the fastening means or the spring elements would press against the inner wall of the outer shaft tube, specifically without the latch elements coming loose from the openings of the inner shaft. Release of the insulation insert from the inner shaft is thus possible only when the inner shaft is pulled with the insulation insert out of the outer shaft. However, since the shafts are always positioned one inside the other during the treatment, the insulation insert cannot accidentally come loose from the inner shaft during the treatment or during the operation.

It is further conceivable that further components can be guided or conveyed through the inner shaft and/or the insulation insert, for example an optical unit, an electrode instrument or other tools. It is conceivable here that an electrode carrier in particular can also be guided through corresponding guides or receptacles into a wall of the insulation insert.

A method for manipulating a surgical handheld device for solving the stated problem has the measures of claim 16. Accordingly, provision is made that, for connecting the insulation insert according to claim 1 in a handheld device according to claim 10 or for preparing for the use of the handheld device, the insulation insert is first of all coupled with a proximal end to a distal end of the inner shaft, and then the inner shaft is guided with the insulation insert into the outer shaft and, after the use of the handheld device, the inner shaft with the insulation insert is guided out of the outer shaft, and only then is the insulation insert uncoupled from the inner shaft. Only by this sequence of installation or assembly of the handheld device can an unwanted release of the insulation insert from the inner shaft be avoided. The outer shaft secures the insulation insert, in the assembled state, against release of the fastening means. As soon as the outer shaft is moved away from the inner shaft, the insulation insert can be uncoupled from the inner shaft in the known and simple manner.

A preferred illustrative embodiment of the invention is described in more detail below with reference to the drawing, in which:

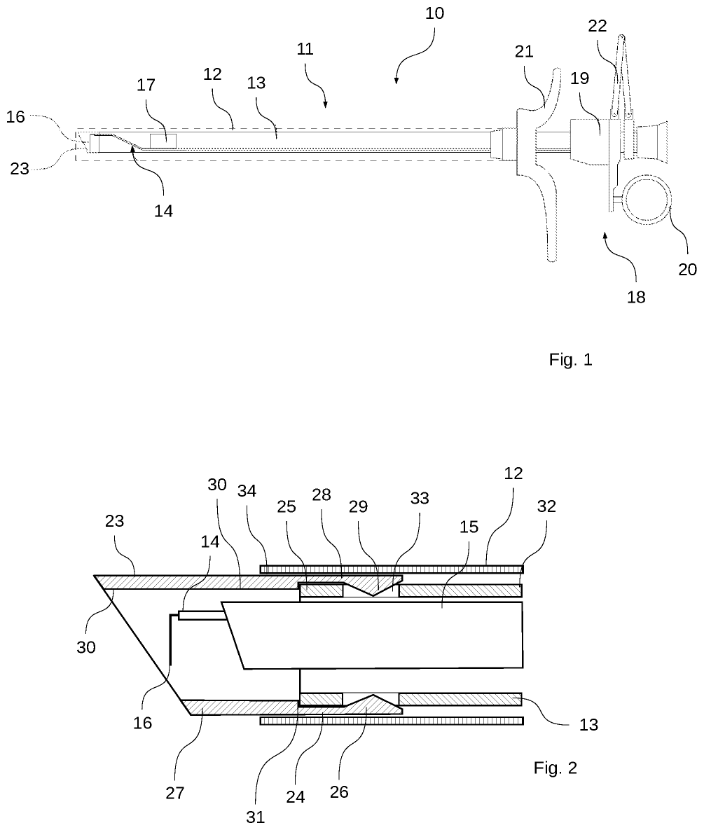

FIG. 1 shows a schematic view of a surgical handheld device, in particular a resectoscope,

FIG. 2 shows a schematic view of a distal end region of the surgical handheld device,

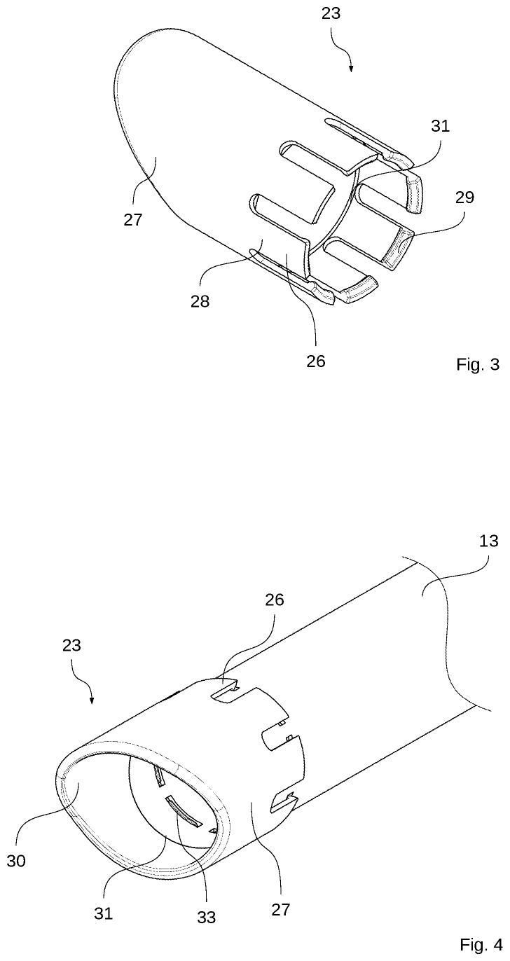

FIG. 3 shows a perspective view of an insulation insert,

FIG. 4 shows a perspective view of the insulation insert with an inner shaft,

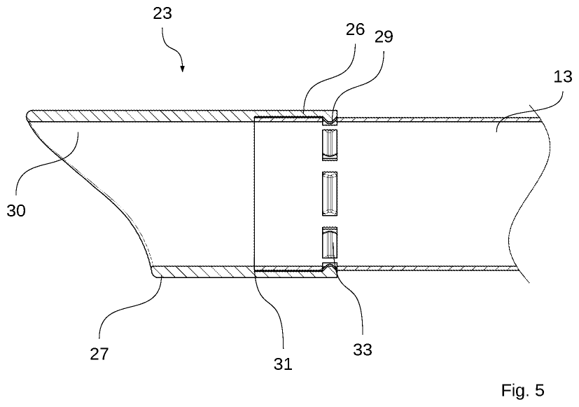

FIG. 5 shows a cross-sectional view of the insulation insert with the inner shaft,

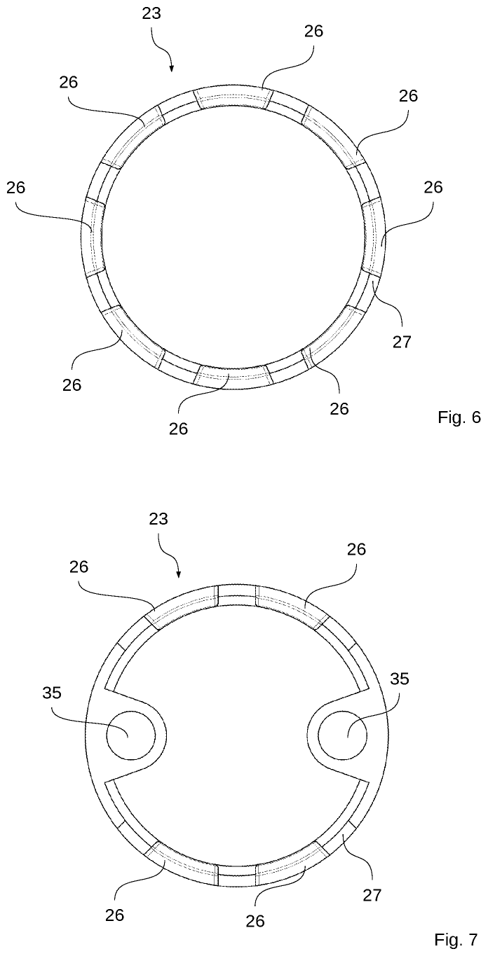

FIG. 6 shows a view of the insulation insert, and

FIG. 7 shows a view of a further illustrative embodiment of an insulation insert.

FIG. 1 shows a schematic cross-sectional side view of a resectoscope 10. It is expressly noted that this resectoscope 10 is shown simply as one possible illustrative embodiment of the claimed surgical handheld device. It is equally conceivable that the surgical handheld device is another instrument different than the resectoscope 10 shown here.

The resectoscope 10 has a resectoscope shaft 11, which comprises an outer shaft 12 or outer tube. A tubular inner shaft 13 runs inside the outer shaft 12. An electrode instrument 14 and an optical unit 15 are shown inside the inner shaft 13. In addition, further elements not shown here can run inside the resectoscope 10, for example a separate irrigation tube and the like.

The electrode instrument 14 has, at a distal end, an electrosurgical tool or an electrode 16. The electrode shown here is formed as a loop, but it can also be configured as a button or the like. It will be seen from FIG. 1 that the electrode instrument 14 is protected by a retention element 17, with a partially circular cross section, against displacements, i.e. displacements deviating from the longitudinal direction of the resectoscope shaft 11. The electrode instrument 14 is mounted longitudinally displaceably in the inner shaft 13. The retention element 17 is of a shape complementary to the inner wall of the inner shaft 13 or to the outer wall of the optical unit 15 and has a partially cylindrical shape.

By actuation of a handle 18, the electrode instrument 14 can be moved in a constrained axial movement in the distal and proximal direction. It can be pushed beyond the distal end of the inner shaft 13 and of the outer shaft 12. The operator is thus also able to manipulate tissue that is located further away from the resectoscope tip. For this purpose, the inner shaft 13 and/or the electrode instrument 14 can also be mounted rotatably about their longitudinal axis. For the manipulation of the tissue, a radiofrequency electric current is applied to the electrode 16.

The resectoscope 10 shown in FIG. 1 has a passive transporter in which, by relative movement of the grip parts 20 and 21 arranged proximally on the resectoscope shaft 11, a carriage 19 is displaced in the distal direction toward the distal, first grip part 21 counter to a spring force applied by a spring bridge 22. In the displacement of the carriage 19 in the distal direction toward the grip part 21, the electrode instrument 14 is displaced in the distal direction in a manner not shown. Upon relaxation of the grip parts 20, 21, the spring force generated by the spring bridge 22 forces the carriage 19 back to its starting position, wherein the electrode instrument 14 is pulled in the proximal direction. Upon the return displacement of the carriage 19, an electrosurgical intervention can be performed with the electrode 16 without manual force from the operator, i. e. passively.

In known surgical handheld devices, the insulation insert is connected fixedly to the shaft. However, since this is unfavorable as regards reuse or as regards sterilizing or cleaning, embodiments also exist in which the insulation inserts are connected releasably to the distal end of a shaft. However, in the known releasable insulation inserts, there is the danger of these coming loose during the operation. On the other hand, if the connection of the insert in the shaft is stronger, releasing the insert proves difficult for an operator.

The insulation insert 23 according to the invention described here is releasably coupled with its proximal end 24 to the distal end 25 of the inner shaft 13 (FIG. 2: only a distal region of the device is shown here). For this purpose, provision is made that the insulation insert 23 has fastening means 26 at its proximal end 25 (FIGS. 3 and 4). These fastening means are designed as snap-fit hooks and are formed from the wall 27 of the insulation insert 23. The fastening means 26 are located, preferably in pairs, at opposite positions on the insulation insert 23 (FIG. 7). It is moreover conceivable that a plurality of fastening means 26 are assigned, at equal angles from each other, to the wall 27 of the insulation insert 23 (FIG. 6). In the illustrative embodiment of an insulation insert 23 shown in FIG. 7, two fastening means 26 are in each case arranged in pairs on opposite sides of the wall 27 of the insert 23. In the illustrative embodiment of an insulation insert 23 according to FIGS. 3 to 6, a plurality of fastening means 26 are located lying opposite each other on the wall 27. The fastening means 26 are at a uniform angle spacing from each other. In this way, it is possible to produce a particularly firm and positionally accurate connection.

In the illustrative embodiment of an insulation insert 23 shown in FIG. 7, the wall 27 has widened parts in which receptacles 35 are arranged for an electrode instrument 14. The receptacles 35 shown here are designed as passages like holes or bore holes, through which the electrode instrument 14 can be guided when the components of the device 10 are joined together.

The fastening means 26 designed as snap-fit hooks are composed principally of a web-like spring element 28 and of a latch element 29 arranged at the free end of the spring element 28. As is shown in the cross section in FIG. 2, this latch element 29 can be wedge-shaped. However, it is also conceivable that the latch element 29 is rounded.

The regions of the wall 27 that are arranged at the proximal end 24 of the insulation insert 23 between the fastening means 26 or the spring elements 28 are on the one hand separated from the fastening means 26, such that the latter can spring freely, and on the other hand these regions have the same thickness as the spring elements 28. A shoulder 31 is thus obtained on an inner face 30 of the insulation insert 23. This shoulder 31 extends in a ring shape around the whole inner face 30 of the insulation insert 23. In the connection of the insulation insert 23 to the inner shaft 13, the insulation insert 23 is pushed onto the shaft 13 until the shoulder 31 abuts against the distal end 25 of the inner shaft 13 (FIGS. 4 and 5).

The inner shaft 13 has openings 33 in its wall 32 at the distal end 25 (FIGS. 4 and 5). These openings 33 are dimensioned and positioned in such a way that the fastening means 26 or the latch elements 29 of the insulation insert 23 snap together when the shaft 13 and the insulation insert 23 are pushed onto each other. As the insulation insert 23 and the inner shaft 13 are pushed one over the other in this way, the spring elements 28 are first of all moved outward by the latch elements 29 and widened until the latch elements 29 snap into the openings 33. In conjunction with the shoulder 31, the insulation insert 23 is located in the optimal position at the distal end 25 of the inner shaft 13. For disassembly, an operator now has to move the latch elements 29 out of the openings 33 or widen the spring elements 28 and pull the insulation insert 23 from the inner shaft 13.

However, in order to ensure, particularly during an operation, that the insulation insert 23 is not accidentally released from the inner shaft 13, the outer shaft 12 is pushed over the inner shaft 13 and partially also over the insulation insert 23 (FIG. 2). According to the invention, the internal diameter of the outer shaft 12 and the external diameter of the insulation insert 23 are such that a gap 34 or an annular space between an inner wall of the outer shaft 12 and the wall 27 of the insulation insert 23 is less than the height of the latch elements 29. The latch elements 29 are fixed in the openings 33 by these narrow gaps 34 or by this small clearance. Even if a mechanical force is exerted on the insulation insert 23, the fastening means 26 or the spring elements 28 cannot be widened in such a way that the insulation insert 23 comes loose from the inner shaft 13. This enveloping action of the outer shaft 12 around the insulation insert 23 has the effect that the insert 23 remains fixed in its orientation relative to the inner shaft 13. The operator can release the insulation insert 23 from the inner shaft 13 only when the outer shaft 12 has been removed again from the inner shaft 13.

In a preferred illustrative embodiment, provision can be made that the insulation insert 23 has a plurality of fastening elements 26, and the inner shaft 13 accordingly has a plurality of openings 33. Besides the shape of the insulation insert 23 shown here, other shapes are also conceivable, for example a straight distal end.

1. An insulation insert for a surgical handheld device, the tubular insulation insert being able to be releasably coupled with a proximal end region to a distal end of a tubular shaft, wherein the proximal end region of the insulation insert has at least two resilient fastening means, which each have a latch element pointing into an interior of the insulation insert.

2. The insulation insert for a surgical handheld device as claimed in claim 1, wherein two or four or six or more fastening means are arranged lying opposite each other on a circumference of the proximal end region.

3. The insulation insert for a surgical handheld device as claimed in claim 1, wherein three or more fastening means are arranged at an angle of 120° or at equal angles relative to each other on a circumference of the proximal end region.

4. The insulation insert for a surgical handheld device as claimed in claim 1, wherein the fastening means are designed as snap-fit hooks.

5. The insulation insert for a surgical handheld device as claimed in claim 4, wherein the snap-fit hook has a tab-like spring element, which is formed out from a wall of the proximal end region of the insert, the latch element being arranged at a free end of the spring element.

6. The insulation insert for a surgical handheld device as claimed in claim 5, wherein the spring element has a reduced thickness in relation to the wall of the insulation insert.

7. The insulation insert for a surgical handheld device as claimed in claim 1, wherein the wall of the insulation insert, in the proximal end region or in the region of the fastening means or between the fastening means, has a reduced thickness in relation to the wall of the insulation insert.

8. The insulation insert for a surgical handheld device as claimed in claim 1, wherein the fastening means are arranged in pairs next to each other, and the pairs are arranged lying opposite each other on a circumference of the proximal end region.

9. The insulation insert for a surgical handheld device as claimed in claim 1, wherein wedge-shaped latch elements have a height that corresponds to the wall thickness of the wall of the insulation insert.

10. A surgical handheld device, with an inner shaft or shaft tube and with an outer shaft or outer tube, and with an insulation insert as claimed in claim 1, the insulation insert being able to be coupled releasably to the inner shaft, and the outer shaft being movable over the inner shaft and the insulation insert.

11. The surgical handheld device as claimed in claim 10, wherein an outer circumference of a proximal end region of the insulation insert is dimensioned in such a way that it can be pushed over a distal end of the inner shaft.

12. The surgical handheld device as claimed in claim 10 wherein a wall of the inner shaft has openings in which latch elements of fastening means of the insulation insert engage for releasable coupling of the insulation insert to the inner shaft.

13. The surgical handheld device as claimed in claim 10, wherein a distance between an inner face of the outer shaft and the outer face of the insulation insert is less than the height of latch elements of the fastening means of the insulation insert.

14. The surgical handheld device as claimed in claim 10, wherein further components can be guided or conveyed through the inner shaft and/or the insulation insert.

15. A method for manipulating a surgical handheld device, as claimed in claim 10 with an inner shaft or shaft tube and with an outer shaft or outer tube, and with an insulation insert to prepare the handheld device for use, the insulation insert is first of all coupled with a proximal end to a distal end of the inner shaft, and then the inner shaft is guided with the insulation insert into the outer shaft, and, after the use of the handheld device, the inner shaft with the insulation insert is guided out of the outer shaft, and then the insulation insert is uncoupled from the inner shaft.
